(19) United States
(12) Patent Application Publication
(10) Pub. No.: US 2018/0200062 A1
(43) Pub. Date: Jul. 19, 2018

(54) IMPLANT AND METHOD FOR COATING AN IMPLANT MAIN BODY

(71) Applicant: JOSSI HOLDING AG, Islikon (CH)

(72) Inventors: Andreas MEYENHOFER, Schlattingen (CH); Christian GUGLER, Frauenfeld (CH); Daniel NADLER, Hettlingen (CH)

(21) Appl. No.: 15/737,868

(22) PCT Filed: Apr. 18, 2016
(86) PCT No.: PCT/EP2016/058549

§ 371 Date: Dec. 19, 2017

(30) Foreign Application Priority Data
Jun. 30, 2015 (EP) 15174449.7

(51) Int. Cl. A61F 2/30; A61F 2/34

(52) U.S. Cl. CPC A61F 2/30771; A61F 2/34; A61F 2002/3082; A61F 2002/30841; A61F 2002/30904; A61F 2002/30322

(57) ABSTRACT

An implant (1), in particular an acetabular implant, for implantation into a bone has, at least in parts, a surface structure (2) of raised portions (3) and depressions (4). The depressions (4) have zones coated with a porous coating (5). The raised portions are either not coated at all or are coated such that the coating has a thin layer thickness or a higher abrasion resistance in these portions. In this way, an improved abrasion behavior is reached when the implant is inserted into the bone.

The present invention relates to an implant for implantation into a bone and to methods for coating an implant main body in accordance with the preambles of the independent claims.

A multiplicity of surface structures are known for anchoring implants, in particular endoprostheses, into bone. These range from roughened surfaces, via special coatings, to equipping the implant surface with complex structural elements.

It is thus advantageous, for example in the case of hip joint prostheses, if implant parts, in particular the joint socket fitted in the hip bone, are anchored in the bone tissue such that they cannot be ripped out or rotated. While a number of joint sockets are fixed using screws, in others the fastening is effected by means of bone cement, while further joint sockets are held directly in the bone by being hammered in or screwed in.

In order to enhance the rip-out behavior and the torsional strength of a joint socket fastened by means of bone cement, by being screwed in or by being hammered in, the surface of such a joint socket is usually provided with a rough surface structure. It is thus possible, for the most part by cutting methods, to work structural elements into the surface which permit a uniform distribution of the forces which arise, both upon insertion and also during later loading of the joint socket, and thereby prevent rotation or even ripping out. In the case of the implant being hammered in, structural elements moreover make it possible for the bone to grow into the interstices thereof.

WO 2012/126944 A1, for example, discloses a hip-joint socket implant in the form of a spherical cap shell, the surface structure of which comprises a multiplicity of structural elements which are formed by in each case a plurality of intersecting grooves with opposite pitch.

U.S. Pat. No. 5,645,593 likewise describes a bone implant with structural elements. However, the interstices between these structural elements are filled entirely with granules. The granules form a porous coating which allows for good secondary fixing by osteointegration.

The presence of structural elements in this case prevents shearing of the porous coating upon insertion of the implant into the bone. Since the depressions between the structural elements are filled entirely with porous material, however, what results is reduced primary fixation of the implant and increased loading of the bone upon insertion of the implant.

U.S. Pat. No. 4,883,491 likewise discloses an artificial hip-joint socket, which however is provided with thread flights for screwing into a bone. Said thread flights are interrupted by regions with a porous coating. However, the porous coating is not protected against shearing as the implant is being screwed in. The porous coating can therefore be abraded during the transport of the implant or when the latter is being handled in the operating theater. The particles which are released as a result can cause damage in the patient's body, and, in the worst case, can pass into the articulation surfaces of the joint prosthesis, where they increase the wear thereof significantly. This can lead to failure of the entire joint implant.

It is the object of the invention to overcome the disadvantages in the prior art.

In particular, it is an object of the invention to provide an improved implant for implantation into a bone which has a surface structure with a porous coating. The intention here is for the implant to allow for improved primary fixation in a bone and at the same time to have a surface with an improved abrasion behavior.

These objects are achieved by an implant having the features in claim 1 and also by a method for coating an implant main body as per claims 9 and 11.

The invention relates to an implant, in particular a hip-joint socket implant, for implantation into a bone. The implant has, at least in parts, a surface structure comprising elevations and depressions. The depressions have zones with a porous coating.

The invention is characterized in that
a) the elevations have uncoated zones which adjoin the coated zones in the depressions,
b) or in that the elevations have coated zones which adjoin the coated zones in the depressions, wherein the layer thickness of the porous coating is smaller on the elevations than in the depressions,
c) or in that the elevations have coated zones which adjoin the coated zones in the depressions, wherein the coating is less porous on the elevations than in the depressions,
d) or in that the elevations have coated zones which adjoin the coated zones in the depressions, wherein the abrasion resistance of the porous coating is greater on the elevations than in the depressions.

In this case, the porous coating extends in a manner following the contour of the depressions.

The terms “elevation” and “depression” here are to be understood in relation to one another. The term “abrasion resistance” in this connection refers to the resistance of the porous coating to mechanical loading, in particular friction. It is determined primarily by its roughness and hardness, but also by the adhesive strength on the implant main body. In experimental terms, the abrasion resistance can be determined by standardized methods, such as for example by abrasive methods or tearing tests.

The porous coating does not necessarily have to be porous over the entire layer thickness. Compacting in places is also conceivable, such that a closed surface is formed. Since coated zones with a low abrasion resistance or a comparatively large layer thickness extend, if at all, only in the region of the depressions of the surface structure, and are therefore protected, the overall abrasion behavior of the porous coating, for example upon insertion of the implant into a bone, is improved considerably. Since the depressions are not filled entirely with porous material, however, the implant nevertheless allows for good primary anchoring in the bone. For this reason, the porous coating always follows the contour of the depressions, but this does not necessarily mean that the coating has to be an exact reflection of the depressions. The essential aspect consists in the fact that an outer contour with depressions and elevations always remains, independently of the thickness of the porous coating.

In the case of the implant, the layer thickness of the porous coating can amount to between 15% and 50% of the profile depth. If the ratio between profile depth and layer thickness lies in this range, what results is a particularly advantageous interaction between primary fixation and secondary fixation. In the present context, the term “profile depth” is understood to mean the average offset between the highest points of the individual elevations and the lowest points of the individual depressions.

In the depressions, the layer thickness of the porous coating can be from 0.1 mm to 0.5 mm. Such a layer thickness allows for good secondary fixing in the bone, with the mechanical stability of the implant being ensured.

The implant can comprise a main structure consisting of titanium, zirconium, niobium, tantalum, or alloys thereof, cobalt-chromium alloy, medical steel, ceramic or polyethylene. With the exception of medical steel, ceramic and polyethylene, the porous coating can also consist of these materials. Materials of this type are widely used for the production of bone implants. They can be processed with methods conventional in the field of the production of generic implants and have an outstanding compatibility with the patient.

In addition, materials of this type can form a supporting structure for the regrowing bone cells, as a result of which natural bone growth is promoted. The use of such materials can accordingly support the healing process of the bone following implantation and also the formation of a stable connection between the implant and the bone. In particularly preferred cases, said materials can even enter into a direct, functional, structural bond with the living bone tissue. This achieves an extremely solid anchoring of the implant in a bone.

The porous coating can be a particle coating and can preferably have particles which are spherical, cubic or cylindrical in shape. Particle coatings have an advantageous porosity. In addition, reliable methods for applying particle coatings to implant main bodies are known in the prior art, such as for example the atmospheric plasma spraying method (APS) or the vacuum plasma spraying method (VPS). By choosing the particle shape, it is possible for the implant surface to be matched to the respective area of use.

The porous coating can have particles, in particular coarse and fine particles, with an average size of 200 μm to 500 μm. Such a particle size gives rise to a porosity which is particularly readily suitable for growth of the bone. By choosing the particle size, it is possible for the implant surface to be matched to a respective use.

The porosity of the coating can be set in such a manner that it is greater at the base of the depressions than in the region of the side flanks of the elevations or on the elevations. This distribution of the porosity further avoids abrasion of the particles upon insertion of the implant into a bone.

Furthermore, the invention relates to a method for coating an implant main body, in particular for producing an implant described above, the method comprising the steps of
providing an uncoated main body, wherein the main body has, at least in parts, a surface structure comprising elevations and depressions,
selectively applying a porous coating, in such a manner that coated zones in the region of the depressions adjoin uncoated zones in the region of the elevations, and in such a manner that the porous coating extends in a manner following the contour of the depressions.

A method of this type has the advantage that, after application of the porous coating, a part thereof does not have to be removed again.

The porous coating can be selectively applied in this case by applying a layer in the region of the elevations which prevents adhesion of the porous coating. Such a layer is also referred to as masking. This variant represents a simple method for selectively applying the porous coating in the region of the depressions which manages without specialized tools.

Selective application of the porous coating is also conceivable, however, by setting parameters of the process, such as for example the beam direction, focus or intensity of a particle beam.

An alternative method for coating an implant main body, in particular for producing an implant described above, comprises the following steps:
providing the uncoated main body, wherein the main body has, at least in parts, a surface structure comprising elevations and depressions,
applying a porous coating in the region with the surface structure,
removing the porous coating in the region of the elevations, in such a manner that
   a) the elevations have coated zones which adjoin the coated zones in the depressions, and in such a manner that the layer thickness of the porous coating is smaller on the elevations than in the depressions,
   b) or in such a manner that the elevations have coated zones which adjoin the coated zones in the depressions, and in such a manner that the abrasion resistance of the porous coating is greater on the elevations than in the depressions,
and in such a manner that the porous coating extends in a manner following the contour of the depressions.

A method of this type represents a particularly simple and cost-effective method for producing an implant described above. Since, in one method step, a porous coating is applied in the region with the surface structure without distinguishing between the regions of the elevations and the regions of the depressions, conventional coating methods which are known very well to a person skilled in the art can be employed in said method.

In this case, the porous coating can be removed in the region of the elevations by shot blasting, shearing, brushing, turning, milling, grinding, barrel finishing, chemical or electrochemical removal. These techniques are widely used and customary in the field of the production of generic implants.

The porous coating can be applied by plasma coating, in particular by an atmospheric plasma spraying method (APS) or a vacuum plasma spraying method (VPS), by precipitation, or additive manufacturing. These methods are widely used in the field, and, depending on the requirements to be satisfied, allow for a reliable, optionally selective, but in any case efficient application of the porous coating.

The aforementioned methods can comprise an additional step, which includes sintering the main body coated with the porous coating. This firstly achieves a high strength of the porous coating per se and secondly, however, also good adhesion on the implant main body.

Further advantages and individual features of the invention become apparent from the following description of two exemplary embodiments and from the drawings.

Schematically:

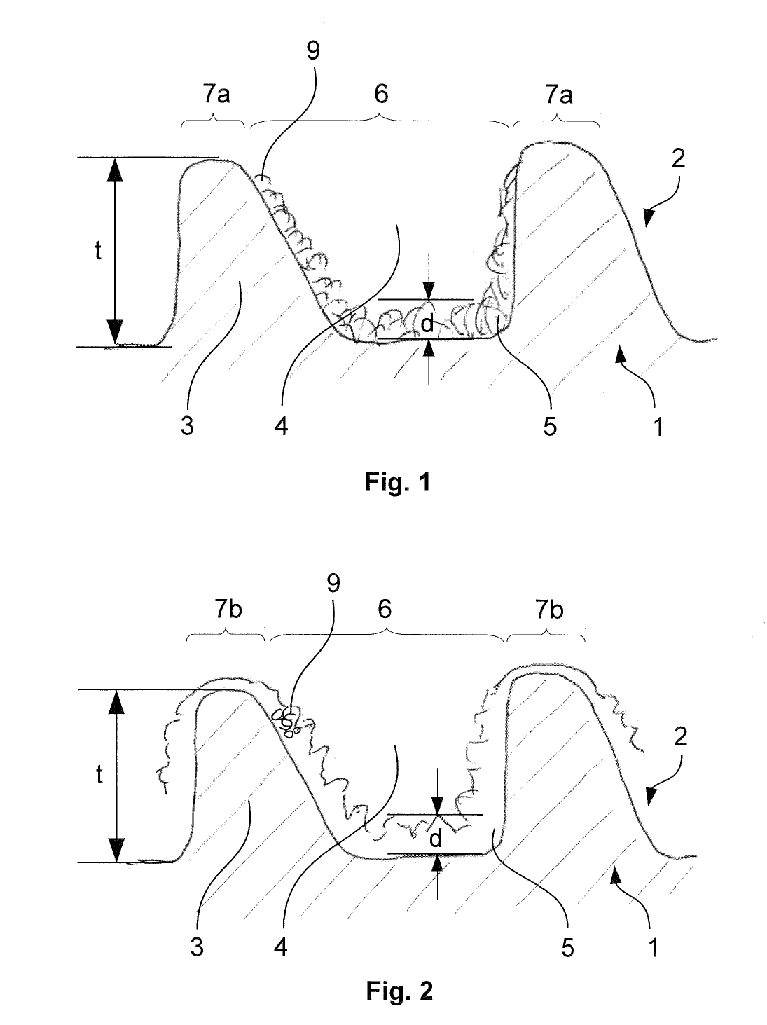

FIG. 1: shows a cross-sectional profile of the surface structure of an implant according to the invention;

FIG. 2: shows a cross-sectional profile of the surface structure of an alternative exemplary embodiment of an implant according to the invention;

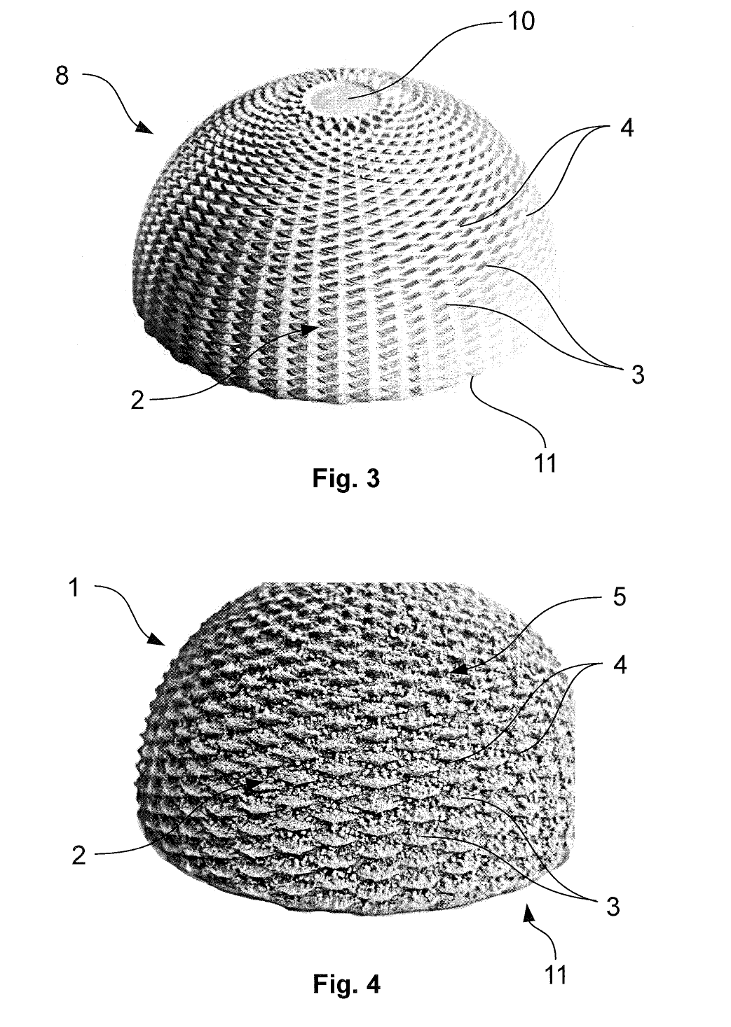

FIG. 3: shows an uncoated implant main body for an implant according to the invention;

FIG. 4: shows an implant according to the invention having a surface structure with a cross-sectional profile as shown in FIG. 1;

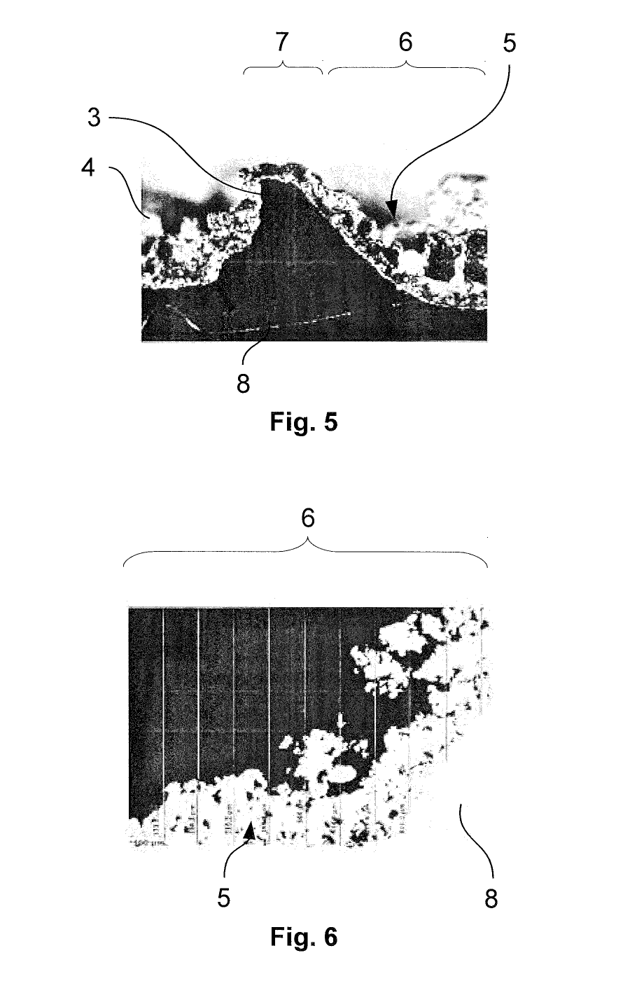

FIG. 5: shows a sectional image of the surface structure of an implant according to the invention;

FIG. 6: shows an enlarged sectional image of the surface structure of an implant according to the invention.

As is apparent from FIG. 1, the surface structure 2 of an implant 1 according to the invention comprises elevations 3 and depressions 4. In the present case, the zones 6 coated with a porous coating 5 extend only over the depressions 4, whereas the elevations 3 have uncoated zones 7a. The surface structure 2 has a profile depth t and the porous coating 5 in the coated zones 6 has a layer thickness d.

As can be gathered from FIG. 2, the exemplary embodiment illustrated therein differs from that shown in FIG. 1 to the effect that both the elevations 3 and also the depressions 4 are coated with a porous coating 5. However, the elevations 3 have coated zones 7b, in which the layer thickness of the porous coating 5 is lower than in the depressions 4. Here, however, the coating can also simply have a lower porosity or a greater compactness than in the depression 4. Therefore, the abrasion resistance of the porous coating 5 is greater in the zones 7b than in the depressions 4. The porous coating 5 illustrated is a particle coating composed of the particles 9.

The implant main body 8 as shown in FIG. 3 was produced by making groove-shaped depressions 4 on a metal blank. This is an implant main body 8 in the shape of a spherical cap shell and having a pole 10 and an equator 11. In addition to the depressions 4 which have already been discussed, the surface structure 2 has elevations 3.

The implant 1 according to the invention which is depicted in FIG. 4 was produced from a main body 8 of the type depicted in FIG. 3, in which case a method as per claim 11 was used. Accordingly, after the provision of an uncoated main body 8, a porous coating 5 was applied in the region with a surface structure, which extends in this case from the pole 10 as far as the equator 11. Thereupon, the porous coating 5 in the region of the elevations 3 was removed again in such a manner that the layer thickness of the porous coating 5 is lower on the elevations 3 than in the depressions 4. The removal of material removes particles of relatively low adhesion, and this leads to a reduction in the layer thickness. As a result, the abrasion resistance of the porous coating 5 is greater on the elevations 3 than in the depressions 4.

FIGS. 5 and 6 show sectional images of the surface structure of implants according to the invention.

As shown in FIG. 5, the elevation 3 has an asymmetrical form in cross section. The coating 5 consisting of titanium particles is distributed differently on the steeply dropping left flank and on the somewhat flatter right flank. The dark regions of the coating correspond to pores between the light titanium particles. It is clearly visible that the layer thickness is greater in the depressions 4 than on the peak of the elevation 3.

FIG. 6 shows, yet further enlarged compared to FIG. 5, an open-pore structure of the coating 5. The light regions of the titanium particles form a rugged structure allowing for the growth of bone substance.

1. An implant (1), in particular a hip-joint socket implant, for implantation into a bone,
wherein the implant (1) has, at least in parts, a surface structure (2) comprising elevations (3) and depressions (4), the depressions (4) have zones (6) with a porous coating (5), wherein
a) the elevations (3) have uncoated zones (7) which adjoin the coated zones (6) in the depressions,
b) or the elevations (3) have coated zones (7) which adjoin the coated zones (6) in the depressions, wherein a layer thickness of the porous coating (5) is smaller on the elevations than in the depressions (4),
c) or the elevations have coated zones which adjoin the coated zones in the depressions, wherein the coating is less porous on the elevations than in the depressions,
d) or the elevations (3) have coated zones (7) which adjoin the coated zones (6) in the depressions, wherein the abrasion resistance of the porous coating (5) is greater on the elevations than in the depressions (4),
and the porous coating (5) extends in a manner following a contour of the depressions (4).

2. The implant (1) as claimed in claim 1, wherein the layer thickness (d) of the porous coating (5), in the depressions (4), amounts to between 15% and 50% of a profile depth (t).

3. The implant (1) as claimed in claim 1, wherein the layer thickness (d) of the porous coating (5) in the depressions (4) is between 0.2 mm and 0.5 mm.

4. The implant (1) as claimed in claim 1, wherein the implant (1) comprises a main body (8) selected from the group consisting of titanium, zirconium, niobium, tantalum, or alloys thereof, cobalt-chromium alloy, medical steel, ceramic or polyethylene.

5. The implant (1) as claimed in claim 1, wherein the implant (1) has a porous coating (5) selected from the group consisting of titanium, zirconium, niobium, tantalum, or alloys thereof, cobalt-chromium alloy.

6. The implant (1) as claimed in claim 1, wherein the porous coating (5) is a particle coating and has particles (9) which are one of spherical, cubic or cylindrical in shape.

7. The implant (1) as claimed in claim 6, wherein the porous coating (5) has particles (9) with an average size of 200 μm to 500 μm.

8. The implant (1) as claimed in claim 1, wherein a porosity of the coating is set in such a manner that it is greater at the base of the depressions (4) than in a region of side flanks of the elevations (3) or on the elevations.

9. A method for coating an implant main body (8), in particular for producing an implant (1) as claimed in claim 1, the method comprising the steps of:
providing the uncoated main body (8), wherein the main body (8) has, at least in parts, a surface structure (2) comprising elevations (3) and depressions (4),
selectively applying a porous coating (5), in such a manner that coated zones (6) in a region of the depressions (4) adjoin uncoated zones (7) in the region of the elevations (3), and in such a manner that the porous coating (5) extends in a manner following the contour of the depressions (4).

10. The method as claimed in claim 9, wherein the porous coating (5) is selectively applied by applying a layer in the region of the elevations (3) which prevents adhesion of the porous coating (5) to a greatest possible extent.

11. A method for coating an implant main body (8), in particular for producing an implant (1) as claimed in claim 1, the method comprising the steps of:
providing the uncoated main body (8), wherein the main body (8) has, at least in parts, a surface structure (2) comprising elevations (3) and depressions (4),
applying a porous coating (5) in a region with the surface structure (2),
removing the porous coating (5) in the region of the elevations (3), in such a manner that:
a) the elevations (3) have coated zones (7) which adjoin the coated zones (6) in the depressions, wherein the layer thickness of the porous coating (5) is smaller on the elevations than in the depressions (4),
b) or in such a manner that the elevations (3) have coated zones (7) which adjoin the coated zones (6) in the depressions, and in such a manner that the abrasion resistance of the porous coating (5) is greater on the elevations than in the depressions (4),
c) or in such a manner that the elevations have coated zones which adjoin the coated zones in the depressions, wherein the coating is less porous on the elevations than in the depressions,
and in such a manner that the porous coating (5) extends in a manner following the contour of the depressions (4).

12. The method as claimed in claim 11, wherein the porous coating (5) is removed in the region of the elevations (3) by shot blasting, shearing, brushing, turning, milling, grinding, barrel finishing, chemical or electrochemical removal.

13. The method as claimed in claim 9, wherein the porous coating (5) is applied by plasma coating, in particular by an atmospheric plasma spraying method (APS) or a vacuum plasma spraying method (VPS), by precipitation, or additive manufacturing.

14. The method as claimed in claim 9, wherein the method additionally comprises the step of
sintering the main body (8) coated with the porous coating (5).
